(19) United States
(12) Patent Application Publication
(10) Pub. No.: US 2017/0105868 A1
(43) Pub. Date: Apr. 20, 2017

(54) Oral Prophylactic Device

(71) Applicant: Christopher Smith, Jamaica, NY (US)

(72) Inventor: Christopher Smith, Jamaica, NY (US)

(21) Appl. No.: 15/285,815

(22) Filed: Oct. 5, 2016

Related U.S. Application Data
(60) Provisional application No. 62/243,357, filed on Oct. 19, 2015.

(51) Int. Cl. A61F 6/00; A61H 19/00

(52) U.S. Cl. CPC A61F 6/00; A61H 19/50; A61F 2006/042

(57) ABSTRACT

A mask is provided to cover the face of a person. The mask can be attached to a person's face through an adhesive strip, which is connected along an interior surface of the mask. An oral region on the mask is defined by a lip, opening, and an elastic tongue sleeve. A power source is placed in the mask to support electrical components. Possible electrical components include an electrical contact for generating sensual shocks, a vibrator for increasing pleasure of sexual acts such as cunnilingus, and an illumination source for adjusting ambient lighting. Such components may be controlled through an interface placed on the mask, or remotely via a wireless component that communications with a control module placed within the mask.

The current application claims a priority to the U.S. Provisional Patent application Ser. No. 62/243,357 filed on Oct. 19, 2015.

### FIELD OF THE INVENTION

The present invention relates generally to an apparatus for safe sex that utilizes a latex construction to protect against venereal diseases that would otherwise be transmitted through oral contact. More specifically, the present invention is manufactured from materials including, but not limited to, latex, polyurethane, silicone, and additional materials. The resulting structure is a mask that covers a user's face in the region around the mouth.

### BACKGROUND OF THE INVENTION

A major health concern in the modern age is the encouragement of “safe sex”.

The population engages in a variety of sexual activities, through which transmission of infections, diseases, and even viruses are possible. Thus, many organizations promote the practice of safe sex, one aspect of which is the use of “protection”. Most commonly, this refers to disposable condoms which prevent direct (i.e. skin-to-skin) contact between a male wearing said condom and his sexual partner.

The present invention seeks to provide a device for protection during oral sex, i.e. in which one person's mouth and tongue are used to stimulate their partner's genitalia. Further, the present invention seeks to enhance sexual experiences by incorporating accessory devices that generate shocks, emit light, create sound, or otherwise stimulate a person's senses. The present invention is a novel device that protects against oral sexually transmitted infections by way of being attached to the face.

The configuration of components for a preferred and potential embodiments of the present invention are described below.

### BRIEF DESCRIPTION OF THE DRAWINGS

FIG. 1 is a front view showing a basic embodiment of the present invention.

FIG. 2 is a rear view showing a basic embodiment of the present invention.

FIG. 3 is a perspective view showing one embodiment of the present invention secured to a person's face.

FIG. 4 is a front view showing one embodiment of the present invention secured to a person's face.

FIG. 5 is a right side view showing one embodiment of the present invention secured to a person's face.

FIG. 6 is a left side view showing one embodiment of the present invention secured to a person's face.

FIG. 7 is a perspective view showing a second embodiment of the present invention secured to a person's face.

FIG. 8 is a front view showing a second embodiment of the present invention secured to a person's face.

FIG. 9 is a right side view showing a third embodiment of the present invention secured to a person's face.

FIG. 10 is a left side view showing a third embodiment of the present invention secured to a person's face.

FIG. 11 is a diagram showing electrical and electronic connections of components of the present invention.

### DETAIL DESCRIPTIONS OF THE INVENTION

All illustrations of the drawings are for the purpose of describing selected versions of the present invention and are not intended to limit the scope of the present invention.

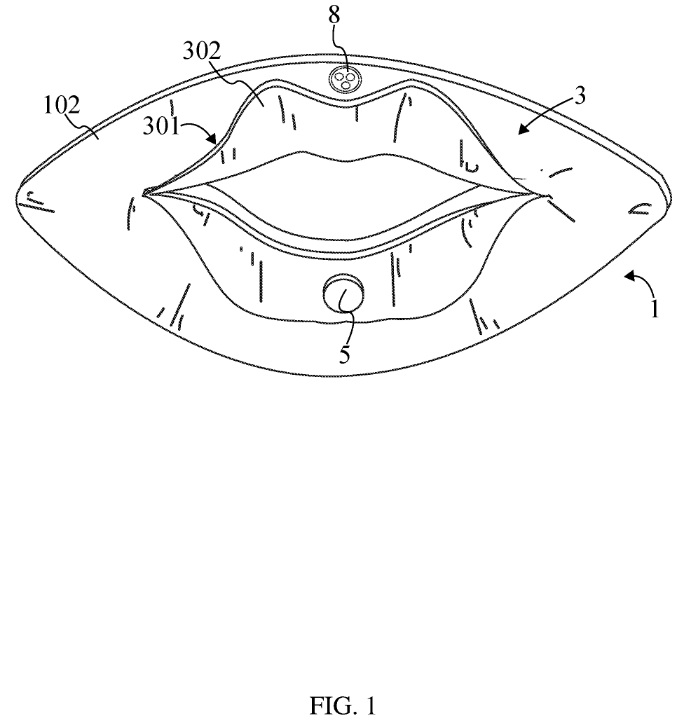

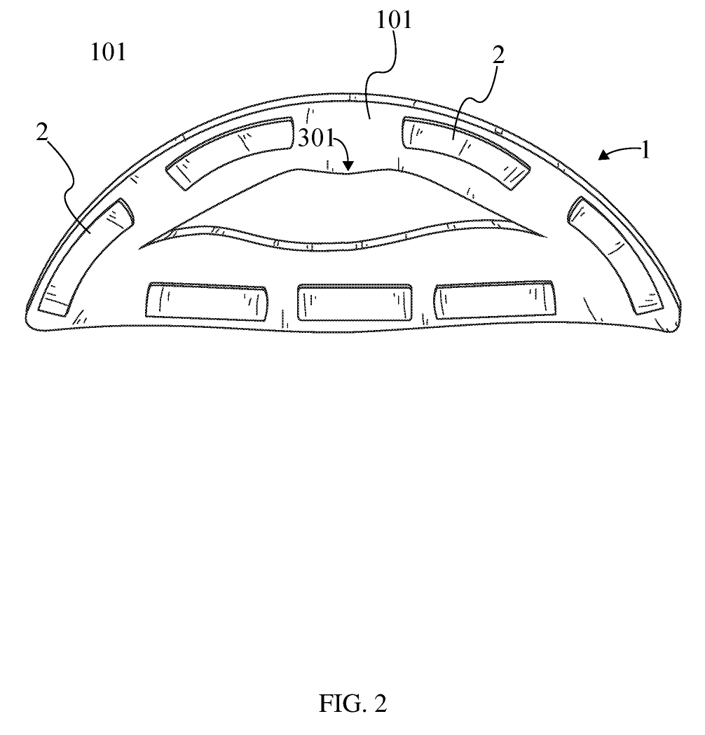

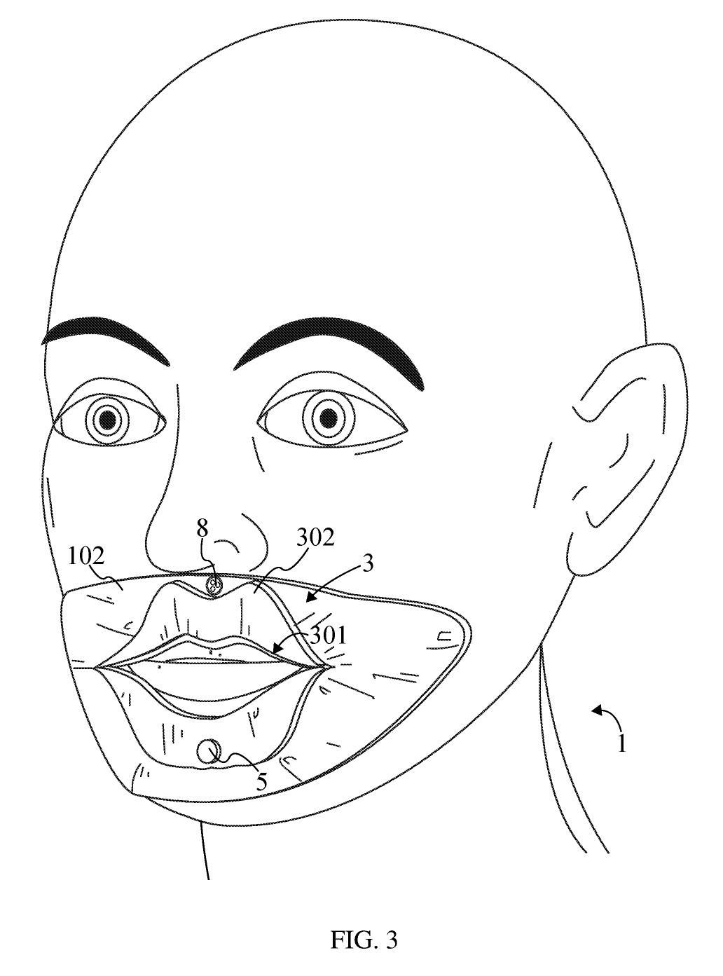

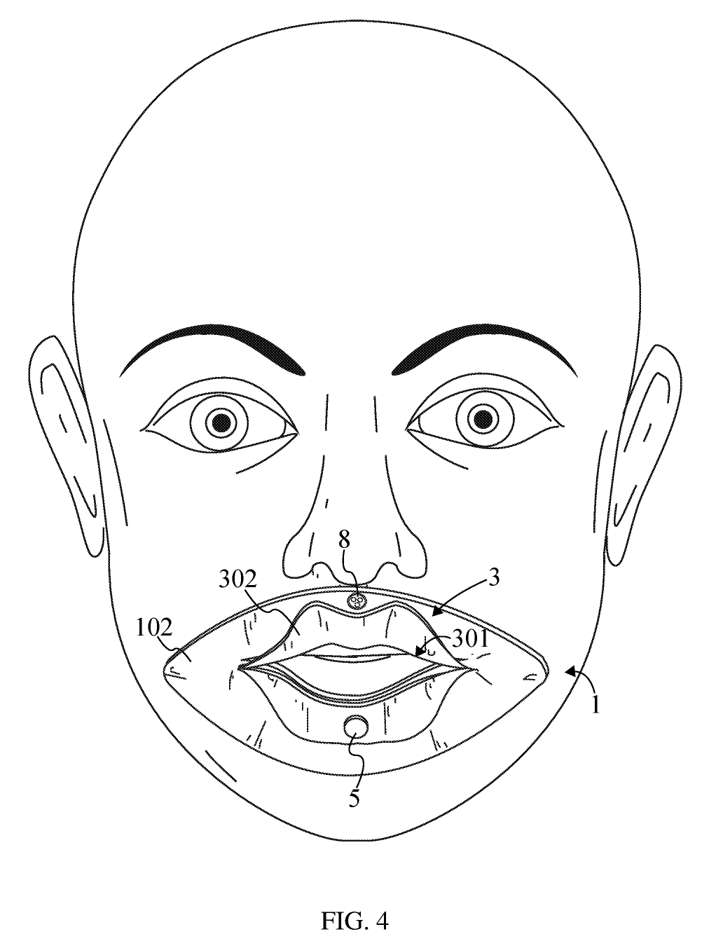

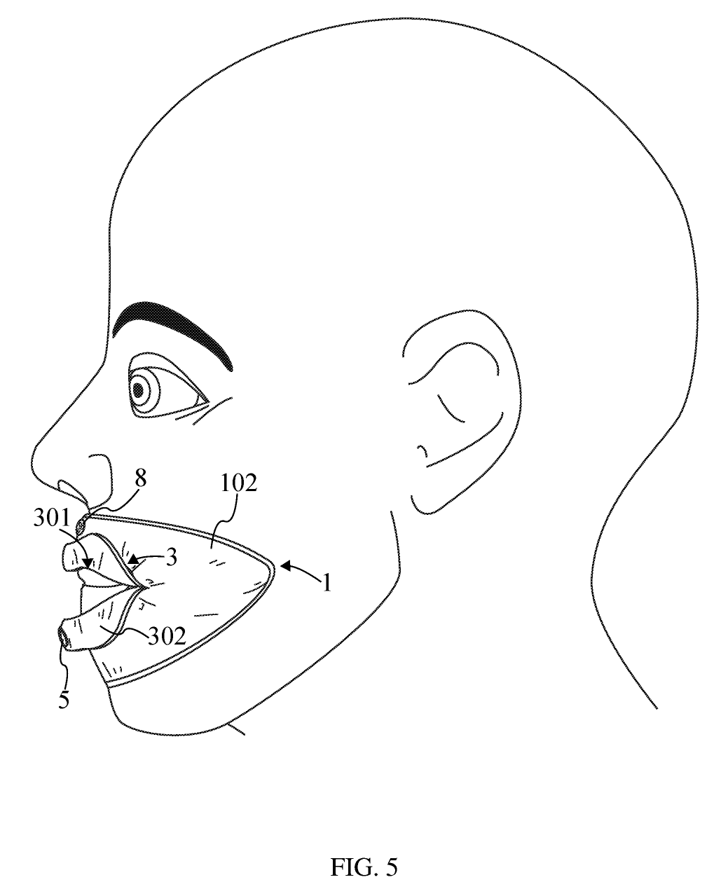

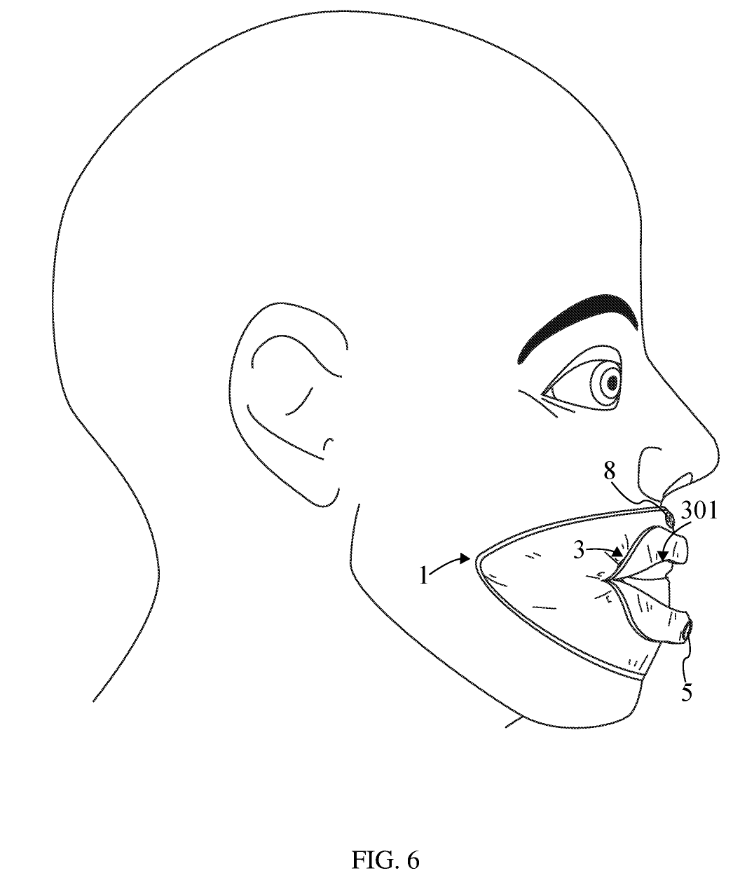

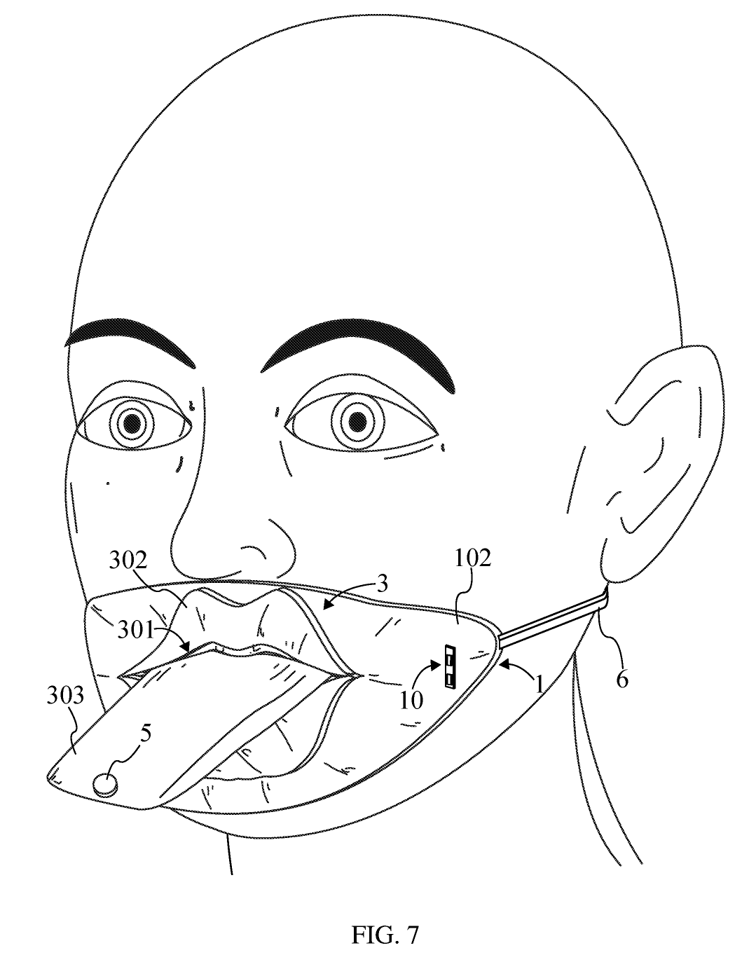

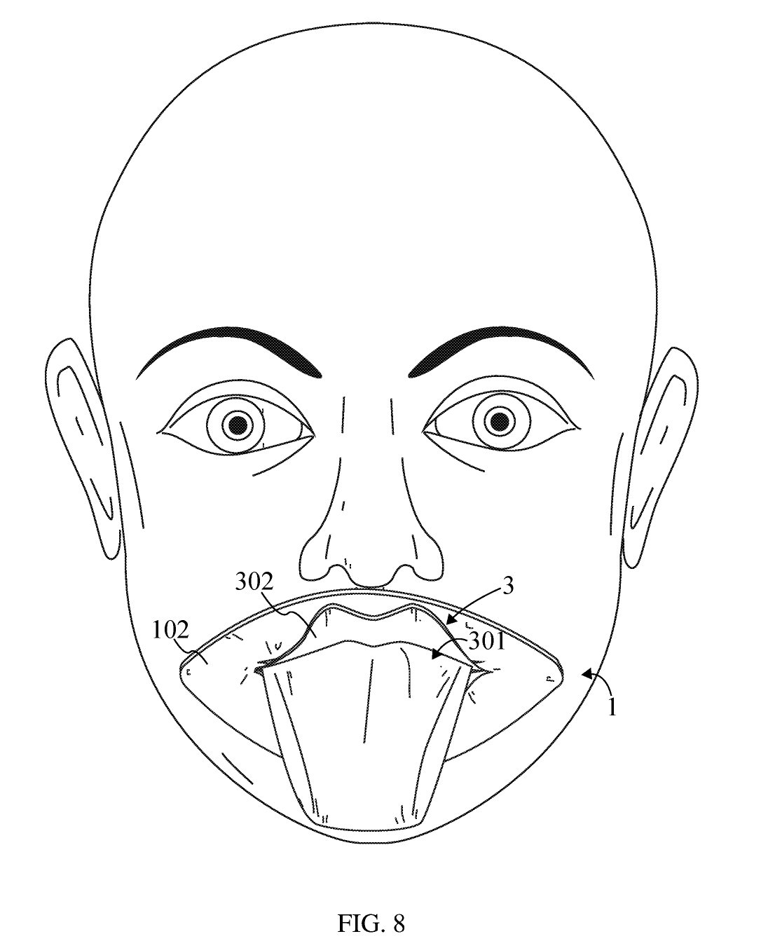

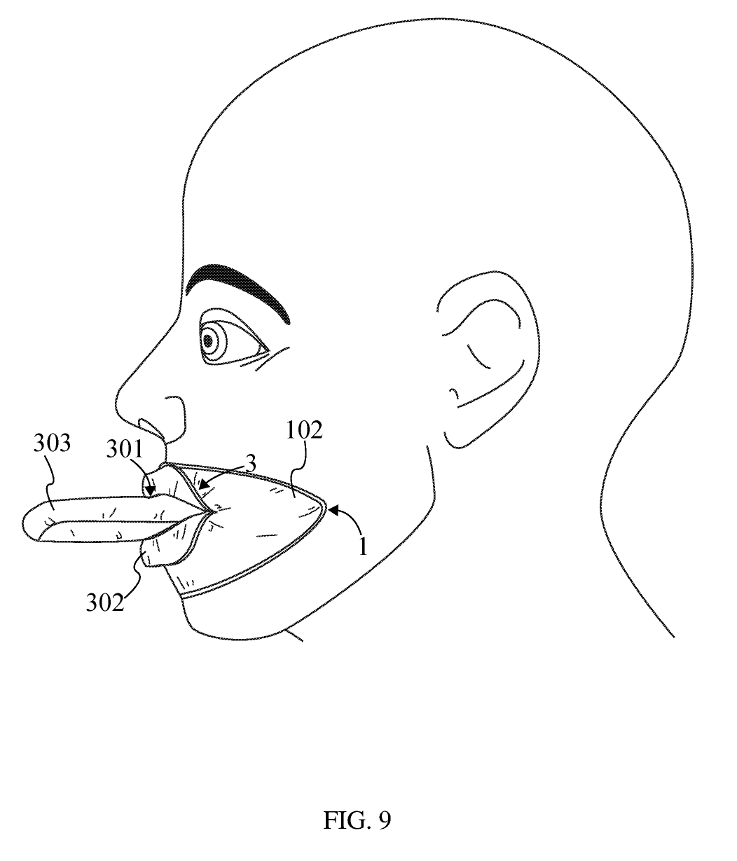

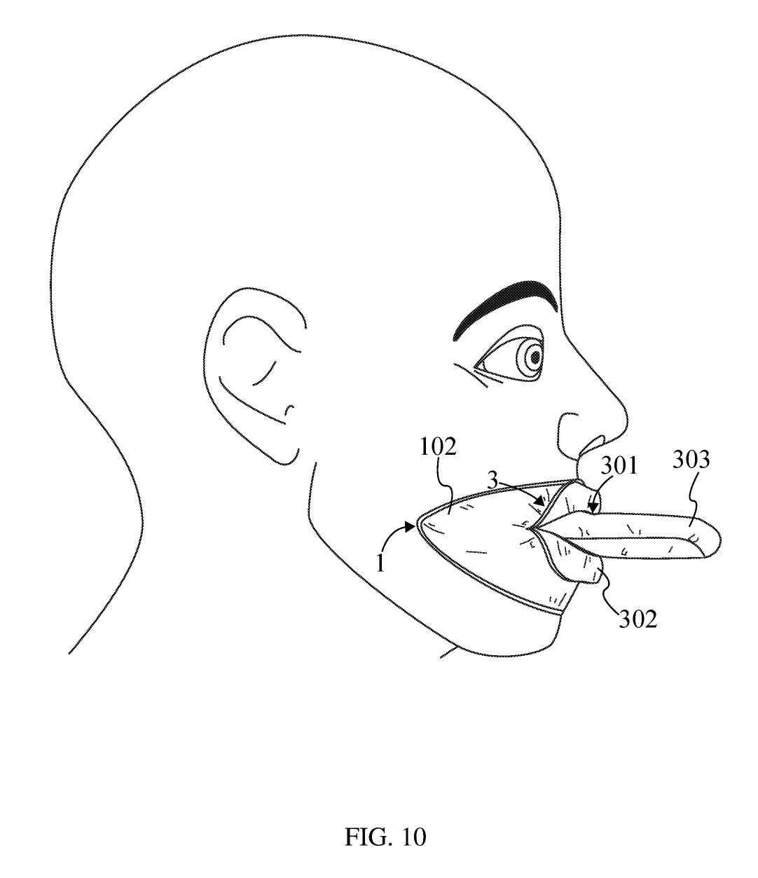

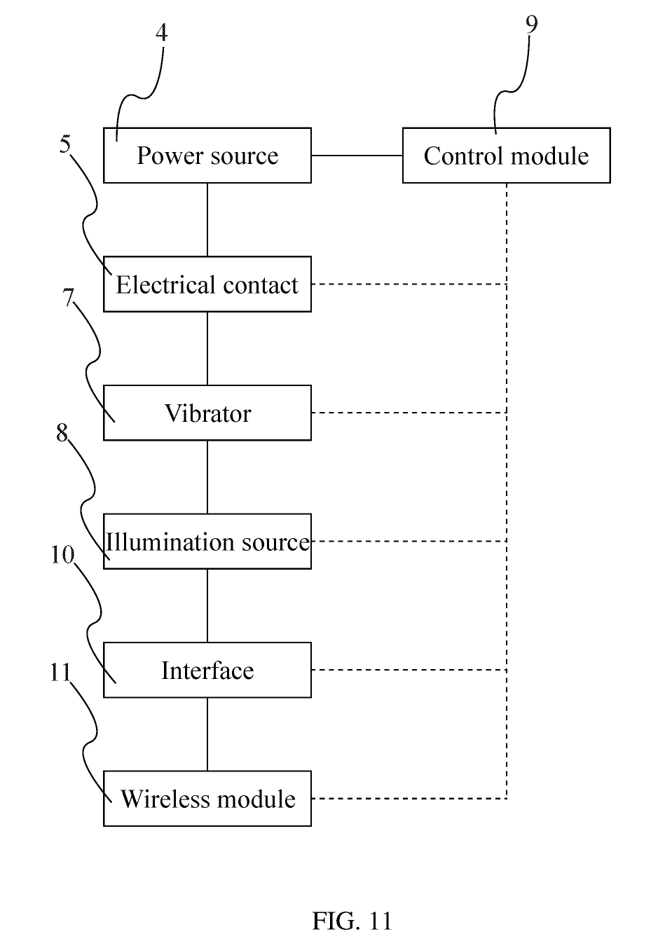

The present invention is an oral prophylactic device that facilitates safe sexual acts, especially of an oral nature. The present invention serves as a barrier to help prevent transmission of sexually transmitted infections (also referred to as “venereal diseases”) that could otherwise occur with direct contact between parties. Additionally, the present invention incorporates several components that enhance the sexual experience. The benefits of the present invention are thus two-fold; protecting against sexually transmitted infections and increasing pleasure derived from sexual acts. The present invention is illustrated via FIG. 1-FIG. 11.

The present invention comprises a mask 1, an adhesive strip 2, and an oral region 3. The mask 1 is the main body of the present invention, which is fitted over at least the jaw of a user's face. The adhesive strip 2 allows for the present invention to easily be secured to a user's face, accomplished by applying sufficient pressure to cause the adhesive strip 2 to stick to a user's face. The oral region 3 is a section of the present invention that is configured to cover a user's mouth. Preferably, the oral region 3 covers the mouth in a manner that prevents direct contact between two parties, for example between a user's tongue and a second person's genitalia.

Preferably, the present invention also comprises a power source 4 and an electrical contact 5, the combination of which serve to pleasurably enhance sexual activities. More specifically, the power source 4 provides electricity to the electrical contact 5, allowing for shocks of varying intensity to be created, helping to ‘electrify’ a sexual encounter.

Describing the above components and corresponding configuration in more detail, the mask 1 comprises an interior surface 101 and an exterior surface 102. The interior surface 101 and exterior surface 102 form opposite faces of the mask 1, with the interior surface 101 being pressed to a user's face and the exterior surface 102 facing away from the user. The oral region 3 comprises an opening 301, a lip 302, and an elastic tongue sleeve 303. The opening 301 is a hole in the mask 1 that a user can stick their tongue through. The hole is outlined by the lip 302, which helps to structurally reinforce the mask 1, as well as serve as a connection point for the elastic tongue sleeve 303. It is noted that the elastic tongue sleeve 303 is not a required part of the present invention; in some embodiments it may be omitted while remaining within the scope of the present invention.

The elastic tongue sleeve 303 is adjacently connected along the lip 302, allowing the elastic tongue sleeve 303 to envelope a user's tongue. This is necessary as it continues to protect against direct contact. Further, the elastic tongue sleeve 303 is non-rigid; it can stretch and generally reform to support movements of a user's tongue. The power source 4, for example a battery, is housed within the mask 1. This convenient positioning allows the power source 4 to easily supply electricity to corresponding components, namely the electrical contact 5.

The electrical contact 5 itself is operatively disposed onto the oral region 3. This allows the electrical contact 5 to engage other items or organisms. For example, an electrifying kiss could be delivered in conjunction with the electrical contact 5, a shock being experienced when the electrical contact 5 is pressed against another person's lips, skin, or generally any body part and region. The exact positioning of the electrical contact 5 can be varied in different embodiments of the present invention.

Elaborating upon the above, in one embodiment the electrical contact 5 could be directly mounted to the elastic tongue sleeve 303. A user could thus apply shocks by moving their tongue to different parts of a receiving person's body. Alternatively, the electrical contact 5 could be mounted on the exterior surface 102 of the mask 1, adjacent to or directly onto the oral region 3. Elaborating upon possible related positions, the electrical contact 5 could be placed on the lip 302 itself or on the exterior surface 102 of the mask 5, adjacent to the lip 302.

Further, in some embodiments of the present invention multiple electrical contacts 5 can be installed. This includes connected multiple electrical contacts 5 to the lip 302 elastic tongue sleeve 303, and the exterior face 102. Correspondingly, if necessary, multiple power sources 4 can be housed within the mask 1 in order to generate sufficient power for the increased number of electrical contacts 5 in any given embodiment.

The adhesive strip 2, which allows the present invention to be secured to a user's face, is preferably positioned adjacent to an edge of the mask 1. Positioning the adhesive strip 2 along a perimeter of the mask 1 helps to create a more secure fit. As earlier noted, the adhesive strip 2 needs to be connected to the interior surface 101. This is because, in order to function properly, the adhesive strip 2 needs to be positioned between a user's face and the mask 1. As with the electrical contact 5 and the power source 4, the present invention is not limited to a single adhesive strip 2. A plurality of adhesive strips 2 can be distributed along the perimeter of the interior surface 101, ensuring that the mask 1 is equally supported at the top portion, the bottom portion, and both side portions.

A further possibility is the substitution or addition of different attachment mechanisms. For example, in one alternative embodiment a clip 6 is operatively disposed on the mask 1. The clip 6, in one version, could be an elastic band that stretches around the back of a user's head. Alternatively, the clip 6 could be a rigid member that clips to the back of a user's head.

Also possible is the application of liquid adhesives, provided said liquid adhesives are safe for human use. These liquid adhesives could be applied along the interior surface 101 of the mask 1, and then pressed against a user's face to dry and set. However, this has a few drawbacks that make it a less than desirable option as compared to the preferred embodiment of the present invention. For one, it requires a user to take the time to apply the adhesive and allow it to set. Second, it has been found that the adhesive leaves a residue on a user's skin after the mask 1 has been removed. This residue requires a user to dedicate additional time to removing the residue and cleaning their faces. Thus, in the preferred embodiment, an adhesive strip 2 is a pressure-sensitive adhesive (e.g. tape) which leaves essentially no residue and facilitates easy attachment and removal of the mask 1.

Other additions are possible with the present invention. Two potential examples include a vibrator 7 and an illumination source 8, which are subsequently elaborated upon. The vibrator 7 is positioned within the elastic tongue sleeve 303, allowing for improved haptic sensations to be experiences by a sexual partner. For example, a user performing oral sex (e.g. cunnilingus) can engage the vibrator 7 to enhance the effect of the oral sex. The illumination source 8 may light up in one or more colors to provide an aesthetically pleasing light display. Both the vibrator 7 and the illumination source 8 can be manually controlled or, alternatively, automatically activated when encountering certain conditions. As bother the vibrator 7 and the illumination source 8 are electronic components, both are electrically connected to the power source 4 to enable their operation. Potentially, a plurality of ridges can be formed on the lip 302 or the elastic tongue sleeve 303 to enhance the effectiveness of the vibrator 7 (resulting in a more pleasurable experience for sexual partner).

To allow for user operation of said accessories, a control module 9 and an interface 10 are provided in certain embodiments of the present invention. The control module 9, similar to the power source 4, is housed within the mask 1. The control module 9 is powered via an electrical connection with the power source 4. The interface 10 is mounted onto the exterior surface 102 of the mask 1. This allows a user to engage the interface 10 in order to operate other components (e.g. the vibrator 7 and the illumination source 8) of the present invention via the control module 9. The specific implementation of the interface 10 can be varied in different embodiment of the present invention. For example, in one embodiment, the interface 10 is a similar on/off switch. In another embodiment, the interface 10 is a button. Other possibilities for the interface 10 include, but are not limited to, dials, sliders, and even touch screens.

In embodiments that include the vibrator 7, the illumination source 8, or ultimately any accessory component, the control module 9 is electronically connected to said accessory component. This electronic connection allows inputs to be transmitted from a user (via the interface 10) to the vibrator 7, for example. Likewise, the control module 9 supports manipulation of the illumination source 8 and other accessory components, when present. Another examples of accessory device includes, but is not limited to, an audio system that emits a sound when a user opens their mouth.

A further possibility, related to the control module 9, is the support of remote instructions. In short, a wireless module 11 can be installed in the mask 1 and communicably coupled with the control module 9. This would allow for the vibrator 7, illumination source 8, and other accessory device to be operated via remote control. As an example, in one embodiment of the present invention a wireless transmitter is fitted into a glove, along with a switch; a person wearing the glove could then flip the switch in order to send transmissions to the mask 1 that engage or disengage the vibrator 7. Said glove could be worn by the same person wearing the mask 1, by a sexual partner, or even by a third party.

Potentially, as earlier referenced, the vibrator 7 and the illumination source 8 could be activated via environmental data. For example, the illumination source 8 could automatically light up when a connected sensor detects light levels that fall below a threshold. Too, the intensity of the illumination could automatically be varied according to the measured amount of ambient illumination. The illumination source 8 could also be configured to activate when triggered by a user opening their mouth, similar to the potential audio system. Another possibility for the illumination source 8 is changing colors based on tactile feedback, for example when the mask 1 is touched by an external entity.

The shape of the mask 1 itself is not restricted by the present invention. In the preferred embodiment, the mask is contoured to match a user's jaw area, terminating below the nose. To allow for a better fit with different contours of individual faces, the mask 1 preferably has at least some level of elasticity. This allows the mask 1 to stretch to better match any given individual's facial characteristics. In some embodiments, it may be desirable to design the mask 1 to resemble a celebrity or popular character. For example, male masks 1 may be designed to resemble the smile of the Joker comic book character, while female masks 1 may be designed to resemble the lips of the Betty Boop cartoon character. Similarly, different color schemes can be applied to the present invention to represent holidays or special occasions. In addition to different colors, flavorings may be incorporated into the mask 1 while remaining within the scope of the present invention.

Although the invention has been explained in relation to its preferred embodiment, it is to be understood that many other possible modifications and variations can be made without departing from the spirit and scope of the invention as hereinafter claimed.

What is claimed is:

1. An oral prophylactic device comprises:
a mask;
an adhesive strip;
an oral region;
a power source;
an electrical contact;
the mask comprises an interior surface and an exterior surface;
the oral region comprises an opening, a lip, and an elastic tongue sleeve;
the opening traversing through the mask;
the opening being delineated by the lip;
the elastic tongue sleeve being adjacently connected along the lip;
the power source being housed within the mask;
the power source being electrically connected to the electrical contact;
the electrical contact being operatively disposed on the oral region; and
the adhesive strip being mounted to the interior surface.

2. The oral prophylactic device as claimed in claim 1 comprises:
the electrical contact being adjacently connected to the tongue sleeve.

3. The oral prophylactic device as claimed in claim 1 comprises:
The electrical contact being adjacently connected to the lip.

4. The oral prophylactic device as claimed in claim 1 comprises:
the adhesive strip being positioned along a perimeter of the mask.

5. The oral prophylactic device as claimed in claim 1 comprises:
a clip; and
the clip being operatively disposed on the mask to secure the mask to a user's head.

6. The oral prophylactic device as claimed in claim 1 comprises:
a vibrator;
the vibrator being positioned within the elastic tongue sleeve;
the vibrator being adjacently connected to the elastic tongue sleeve; and
the power source being electrically connected to the vibrator.

7. The oral prophylactic device as claimed in claim 1 comprises:
an illumination source;
the illumination source being housed within lip, wherein the lip is internally illuminated by the illumination source; and
the power source being electrically connected to the illumination source.

8. The oral prophylactic device as claimed in claim 1 comprises:
a control module;
an interface;
the control module being housed within mask;
the interface being mounted on the exterior surface;
the interface being operatively connected to the control module, wherein user inputs are transmitted to the control module through the interface; and
the power source being electrically connected to the control module.

9. The oral prophylactic device as claimed in claim 8 comprises:
a wireless module;
the wireless module being communicably coupled with the control module; and
the power source being electrically connected to the wireless module.
